(19) United States
(12) Patent Application Publication
(10) Pub. No.: US 2004/0249357 A1
(43) Pub. Date: Dec. 9, 2004

(54) Stretchable fastener

(76) Inventors: Loes Michielsen, Hoogstraten (BE); Bert Bogaerts, Boechout (BE); Michel Coomans, Ham (BE)

Correspondence Address: Cynthia S. Murphy, Renner, Otto, Boisselle & Sklar, LLP, 1621 Euclid Avenue, Nineteenth Floor, Cleveland, OH, 44115-2191, US

(21) Appl. No.: 10/857,086

(22) Filed: May 28, 2004

Related U.S. Application Data
(60) Provisional application No. 60/473,698, filed on May 28, 2003.

(51) Int. Cl. A61F 13/15

(52) U.S. Cl. 604/387

(57) ABSTRACT

A fastener (10) comprising a first attachment section (30) for permanent attachment to a first surface (24) of an anchoring area (20), a second attachment section (40) for permanent attachment to a second surface (26) opposite the first surface (24) on the anchoring area (20), a landing section (50) for permanent attachment to a landing area (22), a fastening section (60) for selective attachment and release from the landing section (50), and a stretchable section (70) between the first attachment section (30) and the fastening section (60).

### RELATED APPLICATIONS

This application claims priority under 35 U.S.C. §119(e) to U.S. Provisional Patent Application No. 60/473,698 filed on May 28, 2003. The entire disclosure of this earlier application is hereby incorporated by reference.

### FIELD OF THE INVENTION

This invention relates generally, as indicated, to a stretchable fastener and, more particularly, to a diaper fastener having an attachment section permanently anchored to a rear portion of the diaper and a fastening section for selective attachment to a front portion of the diaper.

### BACKGROUND OF THE INVENTION

A disposable diaper usually comprises a laminate having a liquid absorbent pad enclosed within a liquid permeable inner shell and a liquid impermeable outer shell. The diaper has a rear portion and a front portion. Fasteners are usually attached to edges of the rear portion and are arranged to provide closure about the wearer upon engagement with respective landing areas on the front portion of the diaper. A typical construction for a diaper fastener comprises two attachment sections for permanently anchoring the fastener to the rear portion of the diaper, and a fastening section for connecting the fastener to a landing area on the front portion of the diaper.

### SUMMARY OF THE INVENTION

The present invention provides a fastener comprising a landing section for permanent attachment to a landing area of a diaper (or other article) and a fastening section for releasable attachment to the landing section. The fastener also comprises a stretchable section between attachment sections and the fastening section. When the fastener is incorporated into a diaper (or another absorbent article), the fastener can provide flexibility, improved dimensional fit, and increased comfort.

More particularly, the present invention provides a fastener comprising a first attachment section for permanent attachment to a first surface of an anchoring area, a second attachment section for permanent attachment to a second surface of the anchoring area, a landing section for permanent attachment to a landing area, a fastening section for selective attachment and release from the landing section, and a stretchable section between the attachment section and the fastening section. The fasteners can be easily manufactured and/or installed by providing a stable stock roll of material which, when laterally cut, provides a plurality of the fasteners.

When the fastener is in a pre-installation condition, the landing section is releasably attached to the second attachment section and the fastening section is releasably attached to the landing section. To install the fastener on the article, the first attachment section is permanently attached to the first surface of the anchoring area and the second attachment section is permanently attached to the second surface of the anchoring area. (The landing section is still releasably attached to the second attachment section and the fastening section is still releasably attached to the landing section.) To deploy the fastener, the landing section is folded away from the anchored attachment sections, with the fastening section being releasably attached thereto. The landing section can then be permanently attached to the landing area, with the fastening section is releaseably attached thereto. To re-open the fastener, the fastening section is released from the landing section.

These and other features of the invention are fully described and particularly pointed out in the claims. The following description and drawings set forth in detail certain illustrative embodiments of the invention. These embodiments are indicative of but a few of the various ways in which the principles of the invention may be employed.

### DRAWINGS

FIG. 1 is a perspective view of a disposable diaper with a pair of fasteners according to the present invention.

FIG. 2 is a side view of the fasteners prior to installation onto the diaper.

FIG. 3 is a perspective view of a roll which can be laterally cut at desired intervals to provide a plurality of the fasteners.

FIG. 4 is a side view of the fastener upon partial installation onto the diaper.

FIG. 5 is a side view of the fastener upon complete installation onto the diaper.

FIG. 6 is a side view of the fastener upon initially securing the diaper onto the wearer.

FIG. 7 is a side view of the fastener as it is being opened.

FIG. 8 is a side view similar to FIG. 2, with a modified version of an attachment section.

FIG. 9 is a side view similar to FIG. 2, with another modified version of an attachment section.

FIG. 10 is a side view similar to FIG. 2, with a modified version of attachment means between two sections.

### DETAILED DESCRIPTION

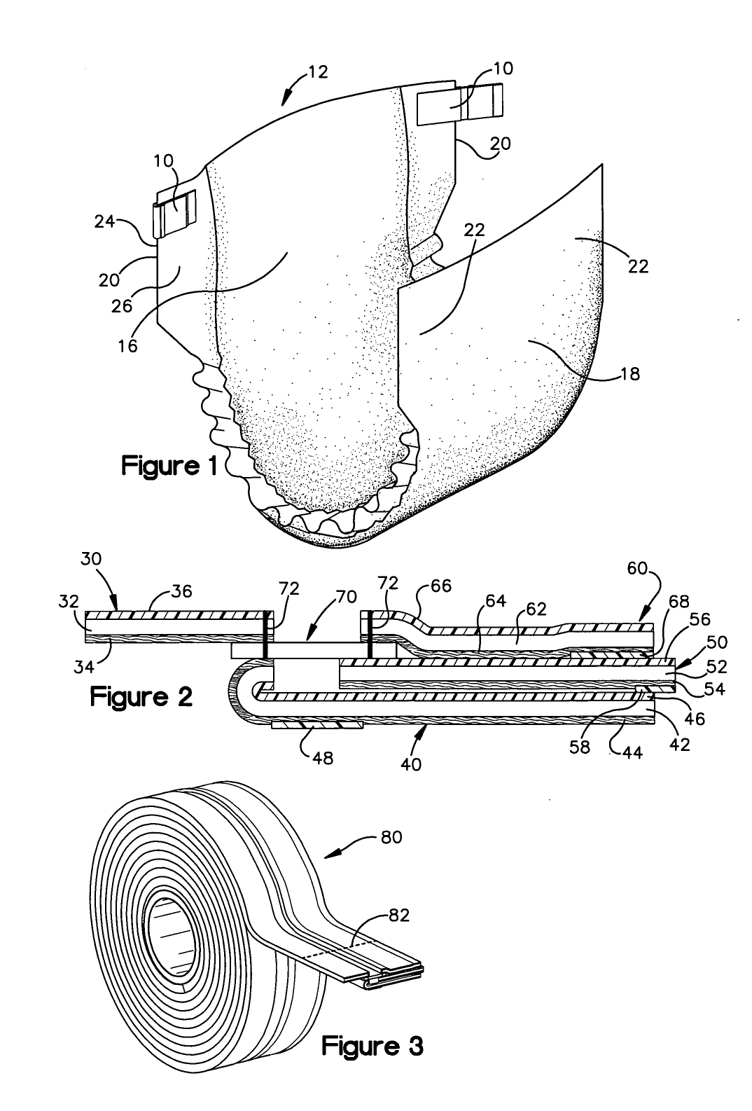

Referring now to the drawings, and initially to FIG. 1, a pair of fasteners 10 according to the present invention are shown installed on a disposable diaper 12. The left hand fastener 10 is shown in a storage condition and the right hand fastener 10 is shown in a deployed condition. As is explained in more detail below, a fastener 10 according to the present invention provides improved flexibility, dimensional fit and/or comfort.

The illustrated disposable diaper 12 comprises a laminate having a liquid absorbent pad enclosed within a liquid permeable inner shell and a liquid impermeable outer shell. The diaper 12 has a rear portion 16 which is intended to cover the wearer's behind and a front portion 18 which is intended to cover the wearer's front. The fasteners 10 are respectively attached to edges 20 of the rear portion 16 and are arranged to provide closure about the wearer upon engagement with respective landing areas 22 on the front portion 18 of the diaper. The edges 20 can be viewed as having an outer surface 24 and an inner surface 26.

Referring now to FIG. 2, a fastener 10 is illustrated isolated from the diaper 12 and in a pre-installation condition. The fastener 10 comprises a first attachment section 30, a second attachment section 40, a landing section 50, a fastening section 60, and a stretchable section 70. With disposable diapers, the length of the fastener 10 in this pre-installation condition is typically in the range of about 40 mm to about 100 mm.

In the pre-installation condition, and in the orientation shown in FIG. 2, the sections 30, 40, 50, 60 and 70 extend generally laterally and are parallel with each other. The stretchable section 70 is positioned laterally between, and just below, the attachment section 30 and the fastening section 60. The landing section 50 is positioned just above the attachment section 40. The stretchable section 70 is positioned vertically between the attachment section 30 and the attachment section 40 and vertically between the fastening section 60 and the landing section 50.

The first attachment section 30 comprises a substrate 32 having an adhesive 34 on one surface and a release coating 36 on the opposite surface. The substrate 32 has a generally rectangular strip shape (although other shapes are certainly possible) and a length representing between about 10% and 60% of the total length of the fastener 10.

The second attachment section 40 comprises a substrate 42 having an adhesive 44 on one surface and a release coating 46 on an opposite surface. The substrate 42 has a generally rectangular strip shape (although other shapes are certainly possible) and is folded in a manner corresponding to the elongated J-shaped shown in the drawings in an exaggerated fashion. (This is known as a “Y” bond in the industry.) The unfolded length of the section 40 represents between about 30% and 100% of the total length of the fastener 10 and its folded length represents between about 3% and 10% of the total length of the fastener 10. A stick-protection pad 48 is provided on the lower surface of the substrate 42 and is aligned with the stretchable section 70.

The landing section 50 comprises a substrate 52 having an adhesive 54 on one surface and a release coating 56 on the opposite surface. The substrate 52 has a generally rectangular strip shape (although other shapes are certainly possible) and a length representing between about 20% and 60% of the total length of the fastener 10. An initial-release finger tab 58 is provided on the substrate's lower surface.

The fastening section 60 comprises a substrate 62 having an adhesive 64 on one surface and a release coating 66 on the opposite surface. The substrate 62 has a generally rectangular strip shape (although other shapes are certainly possible) and a length representing between about 10% and 70% of the total length of the fastener 10. A re-opening finger tab 68 is provided on its lower surface.

The substrates 32, 42, 52 and/or 62 can be made of cloth, kraft paper, cellophane film, non-woven webs, polymeric films (e.g., polypropylene, polyvinyl chloride, polyethylene terephthalate, and polyethylene) or other suitable materials or laminates.

The adhesives 34, 44, 54 and/or 64 can be any conventional adhesive, including pressure sensitive adhesives and non-pressure sensitive adhesives. Suitable pressure sensitive adhesives include acrylic resin and natural or synthetic based rubber adhesives, such as those set forth in U.S. Pat. No. 3,932,328 (the entire disclosure of which is hereby incorporated by reference). With particular reference to the adhesive 64, if it is desired for the diaper 12 to be “re-closeable”, the adhesive should be appropriately selected to accomplish this objective. In-line adhesives (i.e., adhesives applied during the diaper manufacturing stage) can instead be used for the adhesives 34 and 44, and other non-adhesive means (e.g., thermal bonds, magnetic connections, cohesion, mechanical connectors, etc.) could be used instead for any or all of the adhesives. For example, the landing section 50 could comprises a cohesive 56 and the fastening section 60 could comprise a cohesive 64 which cohesively bonds therewith for selective attachment and release of the fastening section 60 from the landing section 50. For another example, and as shown in FIG. 10, the adhesive 64 of fastening section 60 could be replaced with hooks 164 and the release coating 56 of the landing section 50 could be replaced with complimentary loops 156 (or vice-a-versa).

The release coatings 36, 46, 56 and 66 can be a silicone coating, a carbamate coating, or any other coating which promotes deployment of the relevant adjacent section.

The stick-protection pad 48, the finger tab 58, and/or the finger tab 68 can be made of the same or different materials as their base substrates and secured thereto by the adhesives 44, 54, and 64, respectively. However, the functions of these components can be accomplished in different ways or even not be necessary in certain designs. For example, finger tabs can be created by an adhesive-less region on the sections 50 and 60, by rendering adhesive on this region useless, and/or by folding over an end of the substrates 52 and 62.

The stretchable section 70 has a generally rectangular strip shape and a length representing between about 5% and 60% of the total length of the fastener 10. The stretchable section 70 can comprise a piece of elastic material such as a SBL material, a NBL material, an elastomeric film, an elastomeric foam material, etc. In the illustrated embodiment, seams 72 are used to connect the stretchable section 70 to the appropriate other sections of the fastener 10. The seams 72 themselves can comprise adhesive, heat bonds, welds or any other joining means which provides an appropriate junction between the section 30 and the section 70 and/or the section 60 and the section 70. In fact, a strip which integrally incorporates the stretchable section 70 and the attachment section 30 and/or the fastening section 60 would likewise provide appropriate junctions between the relevant sections.

In the illustrated embodiment, the stretchable section 70 is sandwiched between an end of the attachment section 30 and an end of the attachment section 40 and is secured to the attachment section 30 by one seam 72. The other end of the stretchable section 70 is sandwiched between the fastening section 60 and the landing section 50 and is secured to the fastening section 60 by the other seam 72.

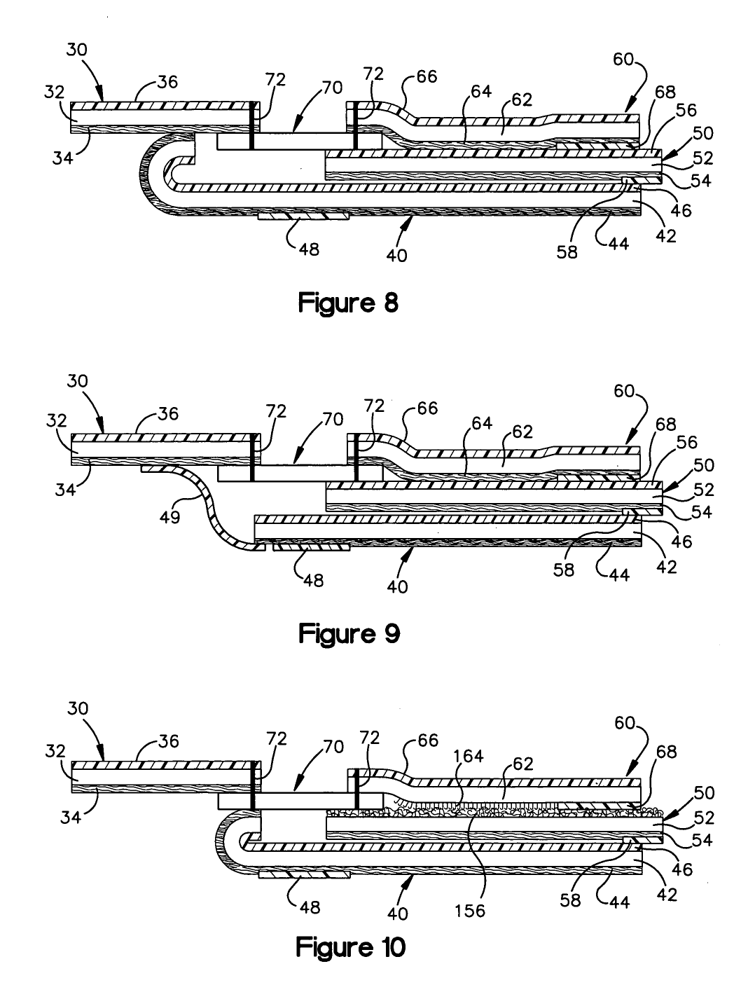

With particular reference to the “sandwiched” position of the stretchable section 70, it may be noted that such precise positioning may not always be possible/practical in certain manufacturing situations and, in any event, is not necessary. For example, as shown in FIG. 8, the J-shaped section 40 can directly contact the adhesive 34 of the attachment section 30. Alternatively, as shown in FIG. 9, the attachment section 40 need not be J-shaped and/or can be secured to the attachment section 30 by a junction strip 49. Other section geometries, section arrangements and/or section-securing schemes are certainly possible with, and are certainly contemplated by, the present invention.

As is best seen by referring briefly to FIG. 3, the fasteners 10 in the pre-installation condition shown in FIG. 2 can be cut from a stock roll 80, preferably in a high-speed efficient manufacturing manner. The width of the roll 80 corresponds to the desired length of the fasteners 10 and the distance of the cut line 82 corresponds to their desired width. It may be noted that the release coating 36 on the attachment section 30 and the release coating 66 on the fastening section 60 prevent adhesion between these sections and the overlying (when in roll form) adhesives 34 and 44. Also, the release coating 56 on the landing section 50 prevents blocking between it and the adhesive 64 on the fastening section 60 when in roll form, pre-installation form, and post-installation form. The pad 48 of the attachment section 40 prevents adhesion of its adhesive 44 to the stretchable section 70 when in roll form. (As such, the pad 48 might not be necessary if the stretchable section 70 does not create sticking issues due to material make-up and/or the positioning of the sections 30 and 60.)

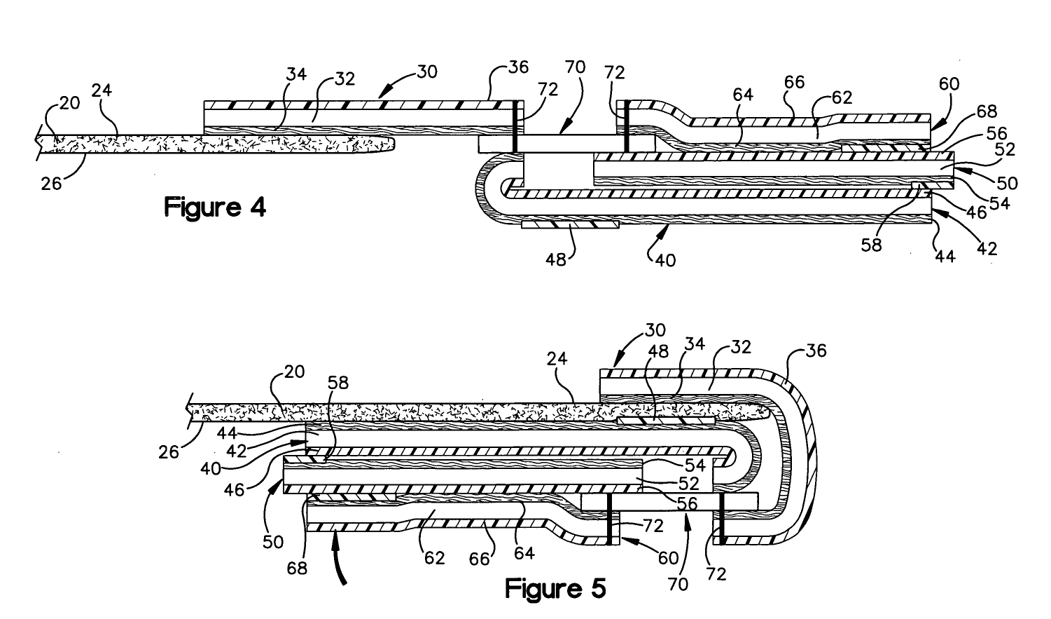

To secure the fastener 10 to the diaper 12, the attachment section 30 is permanently secured to the outer surface 24 of the diaper's edge 20 by the adhesive 34. (FIG. 4.) Then, the attachment section 40, the landing section 50, the fastening section 60, and the stretch section 70 are folded over and the attachment section 40 is permanently secured to the inner surface 26 via the adhesive 44. (FIG. 5). (For the sake of explanation and/or clarity, the gap between the folded attachment sections 30 and 40, and the gap between the elastic section 70 and the attachment section 40 (and/or the diaper 20), may be somewhat exaggerated in some of the drawings. The thin geometry of the sections, and/or the overall thinness of the fastener tape, would result in these gaps being very minimal, if existing at all.)

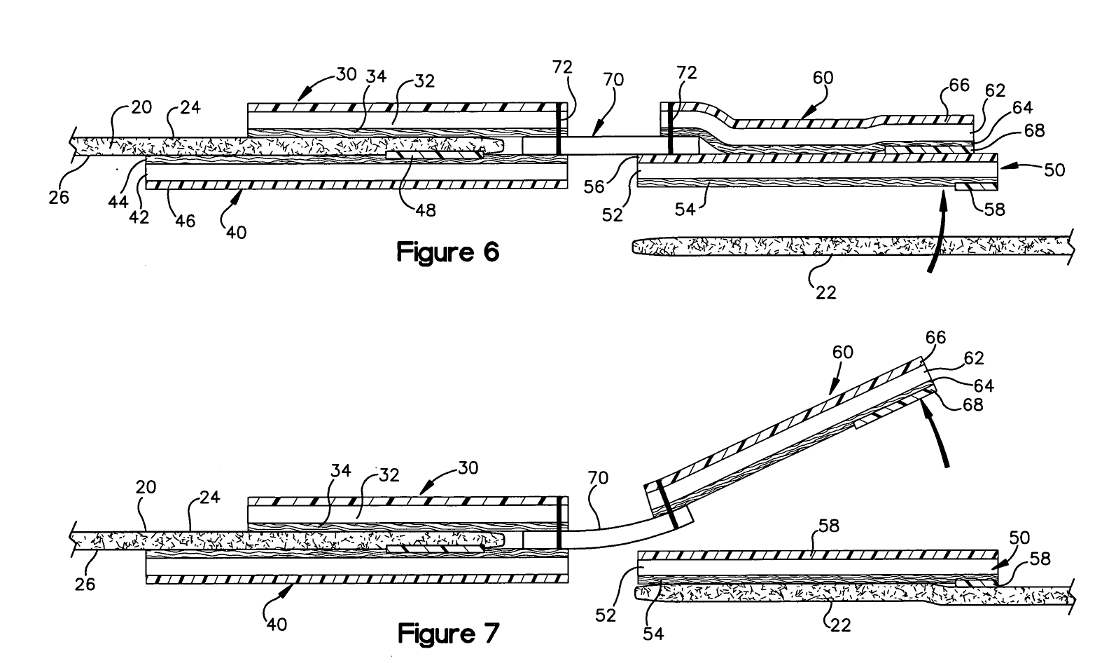

To fit the diaper 12 onto a wearer, the fastener 10 is first placed into a deployed condition by grasping the finger tab 58 and unfolding the landing section 50 (and the fastening section 60 carried therewith) away from the attachment section 40. The front portion 18 of the diaper 12 can then be fitted over the wearer and the landing section 50 permanently secured to the landing area 22 by the adhesive 54. (FIG. 6.) The stretchable section 70 between the attachment section 30 and the fastening section 60 provides an improved dimensional fit and comfort.

To open the fastener 10, the opening finger tab 68 is grasped and the fastening section 60 is folded away from the now-permanently-secured-to-the-diaper landing section 50. (FIG. 7.) Assuming the adhesive 64 and/or the release coating 56 is of an appropriate type, the fastener 10 can be subsequently re-closed by repositioning the fastening section 60 onto the landing section 50. With reference to FIG. 10, the hook-and-loop elements 156 and 164 can provide a similar recloseability feature.

One may now appreciate that the present invention provides a fastener 10 with a built-in landing zone section 50 and a stretchable section 70 that can be mass-produced in an efficient and cost effective manner and provides improved fit and comfort. Specifically, a stable stock roll 80 can be provided which can be cut to produce a plurality of the fasteners 10.

Although the invention has been shown and described with respect to certain preferred embodiments, it is obvious that equivalent and obvious alterations and modifications will occur to others skilled in the art upon the reading and understanding of this specification. For example, although the invention is shown in connection with a disposable diaper for a wearer, the fastener 10 may be employed with non-disposable diapers and/or non-diaper absorbent articles. In fact, the fastener 10 could find application in situations not even remotely involving absorbent articles. The present invention includes all such alterations and modifications and is limited only by the scope of the following claims.

1. A fastener comprising:
a first attachment section for permanent attachment to a first surface of an anchoring area;
a second attachment section for permanent attachment to a second surface opposite the first surface on the anchoring area;
a landing section for permanent attachment to a landing area;
a fastening section for selective attachment and release from the landing section; and
a stretchable section between the first attachment section and the fastening section;
wherein the landing section is releasably attached to the second attachment section and the fastening section is releasably attached to the landing section when the fastener is in a pre-installation condition.

2. A fastener as set forth in claim 1, wherein the fastening section comprises a pressure sensitive adhesive for selective attachment and release of the fastening section to the landing section.

3. A fastener as set forth in claim 1, wherein the landing section comprises a cohesive and the fastening section comprises a cohesive which cohesively bonds therewith for selective attachment and release of the fastening section from the landing section.

4. A fastener as set forth in claim 1, wherein the landing section comprises mechanical fasteners and the fastening section comprises mechanical fasteners which mate therewith for selective attachment and release of the fastening section from the landing section.

5. A fastener as set forth in claim 1, wherein the first attachment section is connected to the stretchable section by a seam and wherein the fastening section is connected to the stretchable section by another seam.

6. A fastener as set forth in claim 5, wherein the seams comprise adhesive, heat bonds, heat welds and/or ultrasonic welds.

7. A fastener as set forth in claim 1, wherein the second attachment section is attached to the elastic section.

8. A fastener as set forth in claim 1, wherein the second attachment section is attached to the first attachment section.

9. A fastener as set forth in claim 8, wherein the second attachment section is attached to the first attachment section via a junction strip.

10. A fastener as set forth in claim 1, wherein a region of the elastic section is exposed between the first attachment section and the fastening section when the fastener is in the pre-installation condition.

11. A fastener as set forth in claim 10, wherein a stick protection pad on the second attachment section is aligned with the exposed region of the elastic section.

12. A stable stock roll comprising a substantially long strip of material which, when laterally cut, provides a plurality of the fasteners set forth in claim 1, in the pre-installation condition.

13. An article including a fastener as set forth in claim 1, wherein the article includes the anchoring area and the landing area.

14. An article as set forth in claim 13, wherein the fastener is in an installation condition whereat:
the first attachment section is permanently attached to a first surface of an anchoring area of the article and the second attachment section is permanently attached to a second surface opposite the first surface of the anchoring area; and
the landing section is releaseably attached to the second attachment section and the fastening section is releaseably attached to the landing section.

15. An article as set forth in claim 13, wherein the fastener is in a deployed condition whereat:
the first attachment section is permanently attached to a first surface of an anchoring area of the article and the second attachment section is permanently attached to a second surface opposite the first surface of the anchoring area; and
the landing section extends away from the attachment sections and the fastening section is releaseably attached to the landing section.

16. An article as set forth in claim 13, wherein the fastener is in an attached condition whereat:
the first attachment section is permanently attached to a first surface of an anchoring area of the article and the second attachment section is permanently attached to a second surface opposite the first surface of the anchoring area; and
the landing section extends away from the attachment sections and is permanently attached to a landing area of the article and the fastening section is releaseably attached to the landing section.

17. An article as set forth in claim 13, wherein the fastener is in a re-opened condition whereat:
the first attachment section is permanently attached to a first surface of an anchoring area of the article and the second attachment section is permanently attached to a second surface opposite the first surface of the anchoring area; and
the landing section extends away from the attachment sections and is permanently attached to a landing area of the article and the fastening section is released from the landing section.

18. An article as set forth in claim 13, wherein the article is a disposable diaper, wherein the anchoring areas are located on edges of a rear portion of the diaper, and wherein the landing area is located on a front portion of the diaper.
